(19) United States
(12) Patent Application Publication
(10) Pub. No.: US 2024/0188944 A1
(43) Pub. Date: Jun. 13, 2024

(54) APPARATUS AND METHOD FOR REMOVING TISSUE

(71) Applicant: ORCA SURGICAL LTD., Caesarea (IL)

(72) Inventors: Roni TALIC, Caesarea (IL); Zvi SHALVI, Hadera (IL)

(73) Assignee: ORCA SURGICAL LTD., Caesarea (IL)

(21) Appl. No.: 18/287,522

(22) PCT Filed: Apr. 28, 2022
(86) PCT No.: PCT/IL2022/050436

§ 371 Date: Oct. 19, 2023

Related U.S. Application Data
(60) Provisional application No. 63/296,194, filed on Jan. 4, 2022.
(60) Provisional application No. 63/181,532, filed on Apr. 29, 2021.

(51) Int. Cl. A61B 10/02; A61B 17/00; A61B 17/3207; A61B 17/3211

(52) U.S. Cl. CPC A61B 10/0266; A61B 17/320708; A61B 17/3211; A61B 2017/00473; A61B 2017/00862

(57) ABSTRACT

A device and a method for selectively peeling a layer of a superficial tissue are disclosed. The device comprising: an elongated head and a flexible handle. The flexible handle comprises at least one component configured to bend the flexible handle upon application of external pressure and return the flexible handle to its original position upon releasing of the external pressure.

### CROSS-REFERENCE TO RELATED APPLICATIONS

This application claims the benefit of priority of U.S. Provisional Patent Application Nos. 63/181,532 filed Apr. 29, 2021 and 63/296,194 filed Jan. 4, 2022, both titled “APPARATUS AND METHOD FOR REMOVING TISSUE”, the content of which are incorporated herein by reference in their entirety.

### FIELD OF THE INVENTION

The invention relates to the field of tissue removal, more specifically the invention relates to apparatuses and methods for tissue removal.

### BACKGROUND

Removal of thin tissue layers is frequently performed during different medical procedures and operations, such as eye operations, vaginal operations, oral operations, cosmetic procedures (e.g., facial peeling, etc.), and the like. In medical procedures, during the removal act, the physician gently moves a blade on the surface of body tissue. The success of such an operation heavily relies on the ability of the physician to control the amount of pressure applied on the blade and the angle at which the blade meets the tissue. Too much pressure or wrong angle and the tissue may be damaged of the tissue removal may be insufficient.

In cosmetics procedures, peeling the epithelial layer (also known as, resurfacing, microabrasion, laser resurfacing, debridement, etc.) may cause regeneration and rejuvenation of the skin. In order to peel the epithelial layer, a professional or the user scrabs the face with an abrasive paste, pastes an acidic paste over the face, or causes shallow burns using a cosmetic laser. All the listed technics may cause damages to deeper layers of the skin as these methods have limited control over the depth of the peeling action. The success or failure of a cosmetic peeling procedure lays heavily on the professionalism of the user performing the procedure and his/her ability to assess the sensitivity of the treated skin.

Accordingly, there is a need for a device that conducts an accurate and controlled tissue removal for any purpose. Such a device may include a blade that allows performing thin tissue layer removal which is less sensitive to the skill level of the user operating the device.

### SUMMARY

The following embodiments and aspects thereof are described and illustrated in conjunction with systems, tools, and methods which are meant to be exemplary and illustrative, not limiting in scope.

Some aspects of the invention are directed to a device comprising: an elongated head and a flexible handle, wherein the flexible handle comprises at least one component configured to bend the flexible handle upon application of external pressure and return the flexible handle to its original position upon releasing of the external pressure.

In some embodiments, the at least one component is selected from: at least a portion of the flexible handle made from an elastic material, a spring, and a joint. In some embodiments, the flexible handle is a replaceable handle. In some embodiments, the flexible handle comprises a connector for detachably connecting the flexible handle to the elongated head.

In some embodiments, the head is disposed with one or more recesses that are configured to engage with the flexible handle, and wherein the recesses are disposed at a proximal end of the head and engaging with the flexible handle comprises having no contact with the distal edges of blades included in the elongated head.

In some embodiments, the elongated head comprises one or more blades positioned substantially in parallel with one or more guiding gaggers and wherein the elongated head has a length ranging between 1-100 millimeters and having a distal end having a length ranging from 1-50 millimeters formed by an elongated edge of the one or more blades. In some embodiments, at least one of the elongated edges is covered with a functional coating, selected from: a lubrication material, a medication, nutritive material, moisturizing material, and an antiseptic material. In some embodiments, the device further comprises another guiding gagger, wherein the blade is a double-sided blade, and wherein the two guiding gaggers are disposed on either side of the one or more blades.

Some additional aspects of the invention are directed to a method for modifying a tissue, comprising: applying external pressure on a flexible handle connected to an elongated head, wherein the flexible handle comprises at least one component configured to bend the flexible handle upon application of external pressure and return the flexible handle to its original position upon releasing of the external pressure.

In some embodiments, applying the external pressure is conducted in motion while moving the elongated head over the tissue. In some embodiments, the at least one component is selected from at least a portion of the flexible handle made from an elastic material, a spring, and a joint.

In some embodiments, wherein the peeled portion of the tissue is the corneal epithelium that does not include the Bowman's layer. In some embodiments, the peeled portion of the tissue is a portion of one of, vaginal tissue, an oral and nasal tissue.

In some embodiments, the method further comprises collecting any peeled tissue and released residue in a funnel of the head. In some embodiments, the released residue is a liquid. In some embodiments, collecting comprises absorbing with an absorbent material at least partially coating the funnel.

In some embodiments, the method further comprises orienting the elongated head with respect to the tissue to define the penetration depth of the elongated edge of the one or more blades into the tissue.

In some embodiments, the elongated head comprises one or more blades positioned substantially in parallel with one or more guiding gaggers and wherein the elongated head has a length ranging between 1-100 millimeters and having a distal end having a length ranging from 1-50 millimeters formed by an elongated edge of the one or more blades. In some embodiments, the method further comprises orienting the one or more guiding gaggers, and the one or more blades substantially parallel to the tangent of the tissue causes the one or more blades to penetrate to a relatively shallow depth and remove a thin layer of the tissue. In some embodiments, the method further comprises orienting the elongated one or more guiding gaggers and one or more blades substantially perpendicular to the tangent of the tissue causing the elongated blade to penetrate to a depth corresponding to the height differential between the one or more guiding gaggers and one or more blades and remove a layer of the tissue corresponding to the penetrated depth.

In some embodiments, applying the external pressure comprises pressing the elongated edge of the one or more guiding gaggers onto the tissue to flatten the surface of the tissue, and enclosing a portion of the flattened tissue within the gap, thereby peeling the tissue at a uniform thickness.

In some embodiments, the method further comprises performing any one of: a refractive eye surgery, treating a myopia disorder, treating a hyperopia disorder, treating an astigmatism disorder, and treating a keratoconus disorder subsequent to the peeling step.

Some additional aspects of the invention are directed to a device comprising: an elongated head and a bent handle bent at a predetermined angle between a central axis of a portion of the handle configured to be held by a user and a connecting surface of a connector connecting the elongated head, wherein the predetermined angle is 90 to 150 degrees with respect to the head and the blades.

In some embodiments, the handle is a replaceable handle. In some embodiments, the connector is configured to debatably connect the flexible handle to the elongated head.

Some additional aspects of the invention are directed to a method for modifying a tissue, comprising: applying external pressure on a bent handle connected to an elongated head, wherein the bent handle is bent at a predetermined angle between a central axis of a portion of the handle configured to be held by a user and a connecting surface of a connector connecting the elongated head, wherein the predetermined angle is 90 to 150 degrees with respect to the head and the blades.

Some additional aspects of the invention are directed to a method for selectively peeling a layer of superficial tissue, comprising: providing a device comprising: an elongated head and a handle, wherein the elongated head comprises one or more blades positioned substantially in parallel with one or more guiding gaggers and wherein the elongated head has a length ranging between 1 to 100 millimeters, and having a distal end having a length ranging from 1 to 50 millimeters formed by an elongated edge of the one or more blades; and removing the layer of the superficial tissue by moving the one or more blades over the designated tissue using the handle.

In some embodiments, the superficial tissue is a portion of facial skin. In some embodiments, removing the superficial layer further includes at least one of removing age spot, removing shallow lesions, and removing post-surgery scars.

In some embodiments, removing the layer further includes at least one of, collecting the removed layer for pathology sampling, and collecting the removed layer for smear squamous cell sampling. In some embodiments, collecting comprises accumulating the layer in a cavity between the one or more guiding gaggers and the one or more blades. In some embodiments, at least one of the elongated edges is covered with a functional coating, selected from: a lubrication material, a medication, nutritive material, moisturizing material, and an antiseptic material.

In some embodiments, the device further comprises more than one guiding gagger, wherein the one or blades are one or double-sided blades, and wherein the one or more guiding gaggers are disposed on either side of the double-sided blade. In some embodiments, the device is a disposable device, and wherein the one or more blades are made from a polymeric material having a shore hardness of between 20 to 90 SHD.

Some additional aspects of the invention are directed to a device comprising: an elongated head and a handle, wherein the elongated head comprises one or more blades positioned substantially in parallel with one or more guiding gaggers and wherein the elongated head has a length ranging between 1 to 100 millimeters and having a distal end having a length ranging from 1 to 50 millimeters formed by an elongated edge of the one or more blades, and wherein the one or more blades are made from a polymeric material having a shore hardness of between 20 to 90 SHD.

In some embodiments, the handle is a flexible handle. In some embodiments, the device further comprises two or more guiding gaggers, wherein the one or more blades are double-sided blades, and wherein the two guiding gaggers are disposed on either side of the double-sided scraping blade.

In addition to the exemplary aspects and embodiments described above, further aspects and embodiments will become apparent by reference to the figures and by the study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-1C illustrate a device for removing a surface layer of a tissue, in accordance with an embodiment;

FIGS. 2A-2I show multiple views of the device of FIGS. 1A-C, where FIGS. 2A-D and FIGS. 2F-I show cross-sections corresponding to those indicated in FIG. 2E, in accordance with an embodiment;

FIGS. 3A-3D together illustrate the device of FIGS. 1A-C coupled to a handle, in accordance with an embodiment;

FIG. 3E is an illustration of a handle according to some embodiments of the invention;

FIG. 3F is an enlarged illustration of a connector for connecting the handle to the elongated head according to some embodiments of the invention;

FIGS. 4A-4C show three additional cross-sectional views of the device of FIGS. 1A-C, in accordance with an embodiment;

FIG. 5 shows a triple-bladed device for removing a surface layer of a tissue, in accordance with an embodiment; and

FIG. 6 is a flowchart of a method for selectively peeling an epithelial layer, according to some embodiments of the invention.

### DETAILED DESCRIPTION

Disclosed herein is a device for removing a surface layer of a tissue, for example, the corneal epithelium (“epithelial tissue”), vaginal tissue, oral tissue, nasal tissue, and the like, and a method for operating the device. In some embodiments, the device may include a flexible handle configured to provide safer operation of the device. The flexible handle may reduce the sensitivity of the removal operation to the pressure and angle, applied by a physician performing the operation, on a blade removing the surface layer of the tissue.

In some embodiments, the device may further include a head formed from at least one blade disposed substantially in parallel with a guiding gagger. Such a head is provided for controllably peeling or scraping the tissue. The blade forms the distal extremity of the apparatus and is configured to peel the tissue, whereas the guiding gagger limits the penetration of the blade, thereby controlling the thickness of the peeled tissue to prevent damage to deeper, tissue layers. The two blades enclose a funnel that collects any removed tissue or fluid. The head may be coupled to a flexible handle, allowing a user or automated robot to maneuver the blades over the tissue.

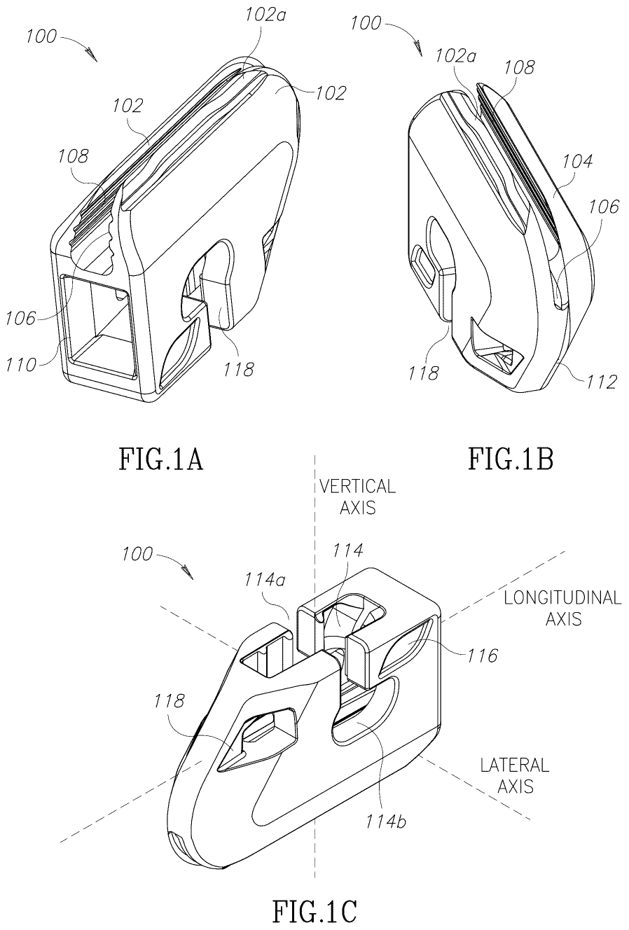

Reference is now made to FIGS. 1A, 1B and 1C which illustrate a device for removing a surface layer of a tissue (e.g., the corneal epithelium), according to an embodiment. An elongated head 100 having substantially trapezoidal side faces is shown, with a longer distal end forming the top of the trapezoid substantially parallel to a shorter proximal end forming the bottom of the trapezoid. To facilitate the description of head 100, three orthogonal axes are indicated in FIG. 1C. The axis labelled ‘vertical axis’ runs from the proximal to the distal ends of head 100. The axis labelled ‘longitudinal axis’ runs from the anterior to the posterior ends of head 100. The axis labelled ‘lateral axis’ indicates the depth or thickness of head 100 and is perpendicular to both the vertical and the longitudinal axes.

The anterior side of head 100 tapers from the longer distal end to the shorter proximal end, forming a rounded acute angle at the anterior distal corner of the trapezoid and an obtuse angle at the anterior proximal corner of the trapezoid. The acute angle may range from 250 to 750. In one embodiment, the acute angle is 40°, or 45°, or 50°. The rounded angle at the anterior distal corner of the trapezoid may bulge outwards such that the anterior extremity is below the distal extremity of the trapezoid, forming a rounded protruding tip at the anterior face of head 100. In some embodiments, the rounded protruding tip protrudes along the longitudinal axis by 0.2 millimeters (mm), or 0.15-2.5 mm, or 0.1-0.3 mm from the anterior edge of the distal face of head 100. In some embodiments, the distance between the anterior extremity of the rounded protruding tip and the distal extremity of head 100 along the vertical axis is 0.2 mm, or 0.15-2.5 mm, or 0.1-0.3 mm. The posterior side of head 100 is substantially perpendicular to the distal end of head 100, forming a substantially orthogonal posterior wall to the parallel top and bottom of the trapezoid. The posterior distal corner of the trapezoid may be rounded along the plane defined by the longitudinal-vertical axes, resulting in a convex surface on the posterior face of head 100.

In some embodiments, the width or thickness of head 100 ranges between, 1-100 millimeters (mm), for example, 2-5 mm, or 2.5-4.5 mm, or 2.7-4 mm, or 2.8-3.5 mm, approximately 3 mm. In some embodiments, the height of head 100 from the proximal to the distal extremity ranges between 1-50 mm, for example, 4-10 mm, or 5-8 mm, or 6-7 mm, or 6.2-6.7 mm.

In some embodiments, elongated head comprises 100 one or more blades 104 positioned substantially in parallel with one or more guiding gaggers 102 and wherein elongated head 100 has a length ranging between 1-100 millimeters and having a distal end having a length ranging from 1-50 millimeters formed by an elongated edge of the one or more blades 104.

The distal end of elongated head 100 is formed by guiding gagger 102 positioned substantially in parallel along the longitudinal axis with at least one elongated blade 104. In some embodiments, blade 104 is made from a polymeric material having a shore hardness of between 20 to 90 SHD (Shore Hardness D).

In some embodiments, blade 104 is made from a polymeric material having a shore hardness of between 30 to 80 SHD. In some embodiments, blade 104 is made from a polymeric material having a shore hardness of between 20 to 80 SHD. In some embodiments, blade 104 is made from a polymeric material having a shore hardness of between 40 to 90 SHD. In some embodiments, blade 104 is made from a polymeric material having a shore hardness of between 40 to 80 SHD. In some embodiments, blade 104 is made from a polymeric material having a shore hardness of between 50 to 90 SHD.

In one embodiment, the angle between the control and blades along the longitudinal axis may range between 5-75°, or optionally between 15-30°. Each of gagger 102 and blade 104 comprises an elongated wedge spanning from the anterior to posterior sides head 100 and having substantially triangular cross-sections, with a narrow, elongated distally disposed edge corresponding to the ‘tip’ of the triangle, and a wider, elongated proximally disposed base corresponding to the ‘base’ of the triangle. The outer wall of each of elongated guiding gagger 102 and blade 104 along the longitudinal axis is convex, forming a curved side of the triangle. The anterior distal corner of control and gagger 102 and blade 104 may taper, forming a rounded anterior distal corner of the trapezoidal face of head 100. Similarly, the posterior distal corner of control and gagger 102 and blade 104 may taper, forming a rounded posterior distal corner of the trapezoidal face of head 100.

The elongated edge of blade 104 extends distally beyond the elongated edge of guiding gagger 102 and is a sharp edge that is configured to cut, peel, or scrape epithelial tissue, forming the distal extremity of head 100. In some embodiments, the external edges of blade 104 form an acute angle of approximately 5°-75°, for example, 10°-50°, or 15°-45°, or 20°-40°, or 25°-35°, or approximately 30°. The elongated edge of blade 104 may extend over the rounded anterior distal corner of head 100, providing a convex surface that comprises the distal extremity and the anterior extremity of head 100.

In some embodiments, at least one of the elongated edges of gagger 102 and blade 104 may be covered with a functional coating, selected from a lubrication material, a medication, and an antiseptic material.

The elongated edge of guiding gagger 102 is disposed below the elongated edge of blade 104 and is configured to press against the tissue without it, forming a barrier that limits the depth of penetration of the sharp edge of blade 104. The external edges of guiding gagger 102 may form a rounded corner having a radius ranging from 0.05 mm-0.1 mm, or one or more flat bands. The elongated edge of guiding gagger 102 may extend over the rounded anterior distal corner of head 100, providing a continuous barrier to the elongated edge of blade 104. In one non-limiting embodiment, the elongated edge of guiding gagger 102 is disposed 0.01-1 mm, for example, 0.05-0.1 mm, or 0.1-0.15 mm, or 0.15-0.2 mm, or 0.2-0.25 mm, or 0.25-0.3 mm, or 0.3-0.5 mm, or 0.05-0.5 mm lower than the elongated edge of blade 104. The elongated distal edges of gagger 102 and blade 104 are separated by a substantially uniform gap (such as may vary between +15% along its length) aligned with the lateral axis, running from the anterior to the posterior ends of head 100 and down the anterior distal rounded corner of head 100, forming the distal and anterior opening to an elongated funnel 106 running from the anterior to the posterior ends of head 100. The width of the gap may range from 0.01-1 mm, for example, 0.1-1.0 mm, or 0.8-0.2 mm, or 0.7-0.3 mm, or 0.6-0.3 mm, or 0.5-0.3 mm. In some embodiments, the width of the gap is 0.4 mm.

The distal edge 102a of guiding gagger 102 may comprise a dull strip, or band extending along the distal surface of guiding gagger 102. Possible widths for the band may range from 0.01-1 mm, for example, 0.05 to 0.25 mm. The band may be warped, having a non-uniform height differential with respect to the sharp edge of blade 104, and resulting in a non-uniform height differential between elongated gagger 104 and blade 102. In one non-limiting embodiment, distal edge 102a bulges higher in the middle section of the elongated edge of guiding gagger 102, resulting in a smaller height differential at the middle sections of gagger 102 and blade 104, and a larger height differential at the peripheral sections of gagger 102 and blade 104. In some embodiments, the height differential between the distal edge of guiding gagger 102a and the distal edge of blade 104 at the bulge is 10%, or 20%, or 30%, or 40%, or 50% smaller than of the height differential at the peripheral sections of gagger 102 and blade 104. The band may be parallel to the proximal base of head 100, or may slope gently, following the external convex surface of guiding gagger 102. In some embodiments, the slope ranges from 5°-75°, for example, 10°-15°, or 15°-20°, or 20°-25°, or 25°-30°, or 30°-35°.

The penetration depth of blade 104 may be defined by a combination of the orientation of the angle of head 100 with respect to the surface of the cornea, the width of the gap between gagger 102 and blade 104 and their respective height differential. Thus, the penetration depth of blade 104 may be controlled by adjusting the angle of control and gagger 102 and blade 104 with respect to the cornea, where orienting gagger 102 and blade 104 substantially parallel to the tangent of the cornea causes blade 104 to penetrate to a relatively shallow depth allowing the removal of a thin layer of epithelial tissue, and orienting gagger 102 and blade 104 perpendicular to the tangent of the cornea, causes blade 104 to penetrate to the height differential between gagger 102 and blade 104, to remove a thicker layer of epithelial tissue corresponding to the penetrated depth. Similarly, the non-uniform height differential between guiding gagger 102 and blade 104 may be leveraged to peel a wider or narrower layer off the cornea, (or any other tissue) wherewith the central section of blade 104 having a smaller differential with guiding gagger 102 results in a thinner peeled slice, and with the peripheral sections of blade 104 having a larger differential with guiding gagger 102 results in a thicker peeled slice.

In some embodiments, the length of the distal end of head 100, forming the top of the trapezoid, may range from 1-50 mm, for example, 4-10 mm, or 5-9 mm, 6-8 mm, or approximately 7 mm. In some embodiments, the length of head 100 ranges between 1-100 mm, for example, 5-13 mm, or 6-12 mm, or 7-11, or approximately 10 mm, having an outwardly protruding rounded anterior tip. In some embodiments, the height of the posterior side of head 100, along the longitudinal axis, forming the orthogonal side of the trapezoid may range from 2-8 mm, or 4-6 mm. In some embodiments, guiding gagger 102 prevents the penetration of blade 104 from exceeding the thickness of the cornea epithelium, ranging between 50-70 microns (μm). In some embodiments, the axis of the height differential between guiding gagger 102 and blade 104 is substantially perpendicular to the axis of the gap between the blades, and the respective hypotenuse is at an angle of 20°, or 25°, or 30°, or 35°, or 40°, or 45°, or 50°, or 55°, or 60°, or 65°, or 65° with respect to the axis of the gap or the axis of the height differential.

Additionally, or alternatively, guiding gagger 104 may be maneuvered to press onto the tissue to somewhat flatten its surface, allowing blade 102 to peel the epithelial tissue at a uniform thickness. The gap between gagger 102 and blade 104 may enclose a portion of the flattened tissue and may be sufficiently small to prevent the pliable tissue enclosed therein from bouncing back to its natural shape.

The oppositely facing inner walls of the elongated wedge portions of gagger 102 and blade 104 enclose funnel 106 that is configured to collect any peeled epithelial tissue and/or other released or secreted residue. Funnel 106 may have a sack-like cross-section, with a narrow elongated distal opening spanning from the anterior to posterior ends of head 100 and formed by the elongated distal edges of gagger 102 and blade 104 corresponding to the gap therebetween, and a wider elongated proximal base formed by the concave inner walls of the wedge portions gagger 102 and blade 104. The sack-like shape allows cut tissue and/or released fluid to withdraw proximally away from the cornea surface. Funnel 106 may be provided with one or more parallel elongated grooves 108 embedded in the inner walls of funnel 106 and extending substantially parallel to the edges of gagger 102 and blade 104 across the length of head 100. Grooves 108 may be disposed at varying heights of the inner walls of funnel 106 and may increase the surface area of the walls of funnel 106, to enhance the capillary action of funnel 106. In one embodiment, funnel 106 is provided with one, two, three, or more grooves 108.

In some embodiment, the base of funnel 106 is 300%, or 400%, or 500%, or 600%, or 700%, or 800%, or 900%, or 1000% wider than the gap forming the distal opening to funnel 106. In some embodiments, the depth or height of funnel 106 from the distal opening to the base ranges from 0.1-0.8 mm, or 0.2-0.7 mm, or 0.3-0.6 mm, or 0.4-0.5 mm.

Funnel 106 may be at least partially coated with an absorbent substance, such as a sponge-like material suitable for absorbing fluid, such as tear solution, water, medication fluid that may be released from the tissue during a cutting, peeling, removing, or modifying procedure and/or any peeled tissue. The absorbent substance may increase the rate of fluid evacuation from the tissue surface during the procedure, allowing for a relatively dry tissue surface that may reduce the chance of uncontrolled sliding of blade 104 of the tissue during the cutting/peeling procedure. In one embodiment, the absorbent substance is disposed within grooves 108 as elongated absorbent funnels. Additionally or alternatively, the elongated base of funnel 106 may be fully or partially coated with the absorbent material, providing an absorbent pocket for any cut tissue and/or released fluid.

FIG. 1A shows a substantially flat posterior end of head 100. The posterior opening of funnel 106 has the sack-like shape as described above, with a narrow distal opening defined by the distal edges of gagger 102 and blade 104, and a wider base defined by the inner convex walls of the proximal wedges of gagger 102 and blade 104. The posterior end of head 100 may be disposed with a window 110 positioned proximally with respect to funnel 106. Window 110 may be square or rectangular in shape, having straight sides and rounded corners.

Referring to FIG. 1B, a tapered anterior end 112 of head 100 is shown, formed by the externally convex anterior walls and rounded anterior distal corners of gagger 102 and blade 104. A portion of the anterior distal rounded corner of blade 104 may be hollowed out, forming a narrow groove comprising an anterior side opening of funnel 106. The narrow anterior opening of funnel 106 may prevent any peeled epithelial tissue collected therein from falling out from the anterior end of head 100, allowing the user to maneuver head 100 while performing the procedure, accordingly. Similarly, the rounded and tapered anterior distal corner of head 100 may allow the user to maneuver head 100 over the tissue during the procedure, to accurately position control and gagger 102 and blade 104 on the surface of the tissue.

Reference is now made to FIG. 1C which shows a nonlimiting example of the bottom proximal face of head 100 having a recess 114 that is configured to engage with a flexible handle, and which will be described in greater detail below. Recess 114 may have any suitable shape for engaging with the flexible handle. In one embodiment, recess 114 forms a cross-shape on the proximal face of head 100, with a longer rectangular portion of the cross disposed longitudinally along the length and enclosed by the proximal face of head 100, and a shorter rectangular portion of the cross disposed horizontally and dividing the proximal face of head 100 into two sections forming two gaps 114a. Gaps 114a may extend to the sides of head 100 to form two hollowed-out lips 114b on each trapezoidal face of head 100. Gaps 114a with lips 114b may penetrate head 100 along the lateral axis, forming hollowed-out windows. Head 100 may be provided with additional recesses, such as recesses 116 disposed on either side of head 100 at the proximal posterior corner of head 100 and recesses 118 disposed on either side of head 100 at the proximal anterior corner of head 100. Recesses 116 may have a parallelogram shape and may be positioned on a flat surface of head 100, whereas recesses 118 may be trapezoidal in shape and may be positioned on the contoured, tapered anterior surface of head 100 wrapping around the external surface of head 100 towards its tapered anterior edge. Recess 116 and/or 118 may penetrate through head 100 along the lateral axis, forming one or more hollowed-out windows.

Gagger 102 and blade 104 may comprise a biocompatible polymer, or metal and may be coated with a hydrophilic material to enhance the collection of any released fluid within chamber 106. Metal blades may be manufactured with a high surface finish, to reduce inflammation and/or an engraving effect on the tissue.

Head 100 may be configured to be used for a limited number of procedures, such as one, two or more procedures, and disposed thereafter. Alternatively, head 100 may be suitable for sterilization and may be used any number of times.

In one embodiment, head 100 has three or more distally disposed blades: two guiding gaggers 102 positioned on either side of one or more central blade 104. The blades may be substantially similar to those described above, with the notable difference that one or more blades 104 are a double-sided blade, allowing for controlled, bidirectional peeling/removing of the epithelium. Guiding gaggers 102 may be symmetrically disposed about central one or more blades 104, having the same features described above, symmetrically positioned about central one or more blades 104. Alternatively, guiding gaggers 102 may have different gaps and differential heights with respect to central one blade 104, allowing for greater variability to control the thickness of the peeled tissue.

Optionally, the contact surface area of the multiple-bladed head is smaller than the contact surface area of the double-bladed head. The number of blades and their respective disposition with respect to each other may be selected to obtain a desired contact surface area with the cornea.

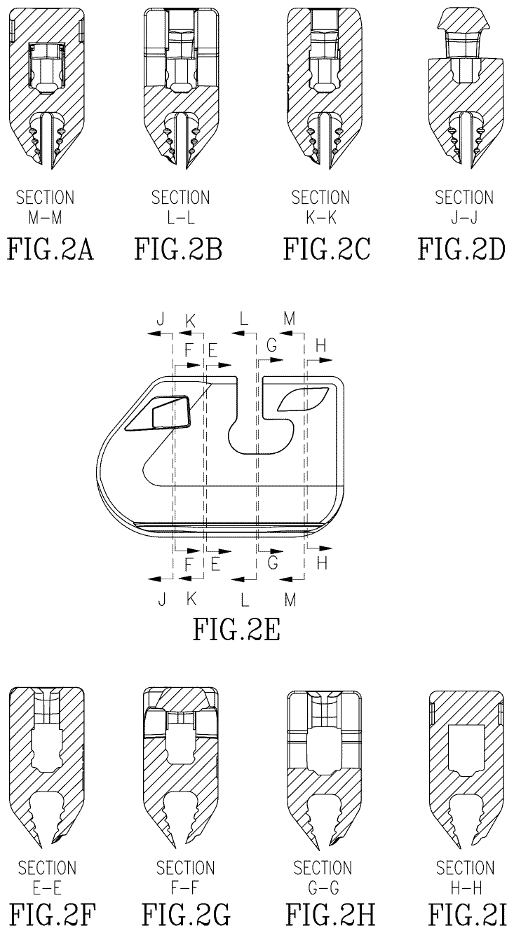

Reference is now made to FIGS. 2A-2I which show multiple cross-sectional views of head 100, corresponding to the slices indicated in FIG. 2E. FIGS. 2A-D show the narrow anterior opening of funnel 106, and FIGS. 2F-2I show the wider sack-shaped posterior opening of funnel 106. The differential height as well as the gap between guiding gagger 102 and blade 104 is clearly shown. FIGS. 2F-I show grooves 108 disposed at varying heights along the inner walls of funnel 106.

FIGS. 2B-2D show multiple cross-sectional views of recess 114. In particular, FIG. 2D is shown engaged with a handle coupled to head 100.

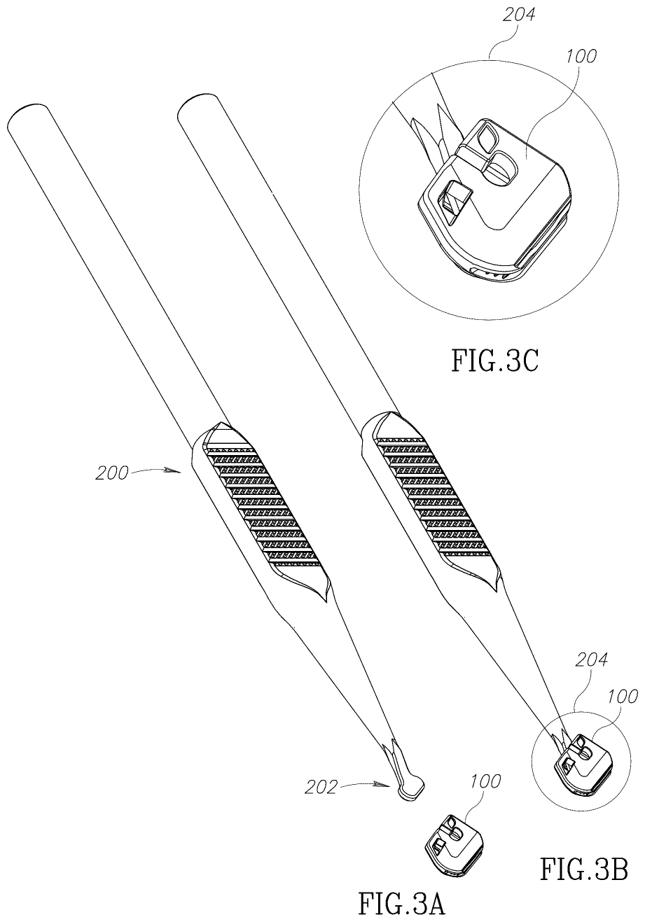

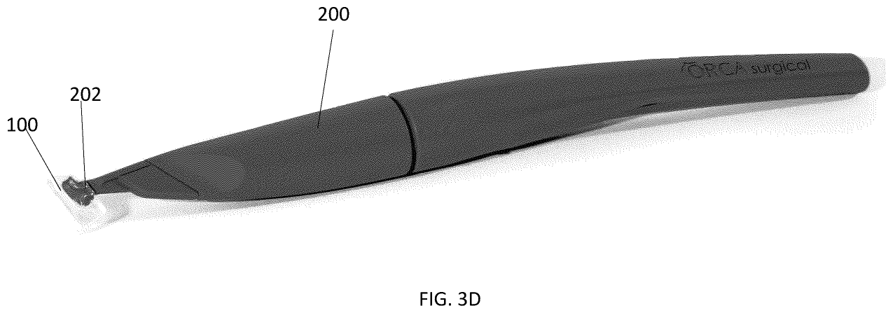

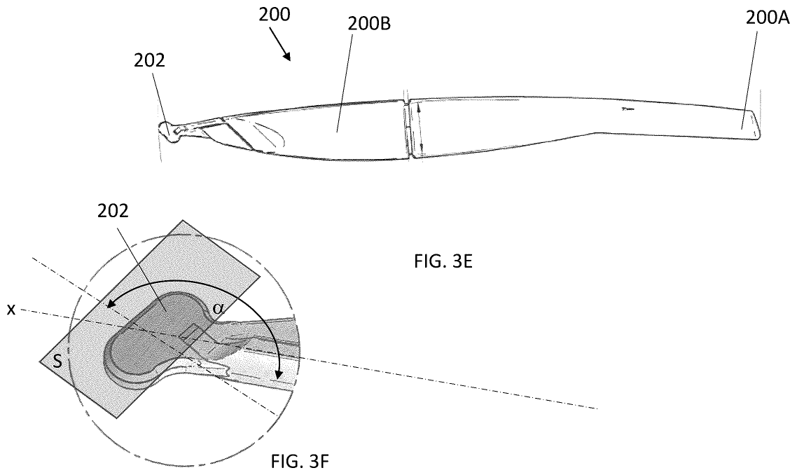

Reference is now made to FIGS. 3A-3D which together illustrate elongated head 100 coupled to a handle 200 which may be a flexible handle, a disposable handle and/or a replaceable handle, that allows the user to maneuver head 100 over a tissue, according to an embodiment. Alternatively, handle 200 may be a bent handle, having two portions 200A and 200B, illustrated in FIG. 3D.

In some embodiments, elongated head 100 has a length ranging between 1-100 millimeters, having a distal end having a length ranging from 1-50 millimeters formed by an elongated edge of blade 104 positioned substantially in parallel with an elongated edge of a guiding gagger 102. In some embodiments, blade 104 is made from a polymeric material having a shore hardness of between 30 to 80 SHD.

FIG. 3A shows handle 200 disengaged from head 100, Handle 200 may be a disposable handle and/or a replaceable handle. FIG. 3B shows handle 200 engaged with head 100, and FIG. 3C shows a close-up 204 of handle 200 engaged to head 100. Handle 200 may include at least one component (not illustrated) configured to bend flexible handle 200 upon application of external pressure and return the flexible handle to its original position upon releasing the external pressure. In some embodiments, the at least one component may be selected from: at least a portion of the flexible handle made from an elastic material, a spring, and a joint. For example, flexible handle 200 may be made from an elastomer or any other polymer that may allow bending flexible handle 200 upon application of external pressure and return flexible handle 200 to original position upon releasing of the external pressure. In another example, a mechanism that includes a joint connecting two portions of flexible handle 200 allows the two portions to pivotally move with respect to each other, when external pressure is applied on flexible handle 200. The mechanism may further include a returning spring for returning the two portions to their original position upon releasing the external pressure.

Handle 200 may taper towards its distal end and may be disposed with a clip-like connector 202 comprising, for example, two flat, disk-like tips that are wider than the tapered portion of handle 200. In some embodiments, connector 202 may detachably connect handle 200 to head 100. Tips 202 may be used to couple handle 200 to head 100 by inserting tips 202 with their flat sides oriented longitudinally with head 100 into, for example, the longer rectangular portion of the cross-shape of recess 114. Handle 200 may be rotated by 90° to align tips 202 with their flat sides oriented with the shorter rectangular portion of the cross-shape of recess 114. Tips 202 may be separated and may couple with head 100 by locking into either end of the longer rectangular portion. Alternatively, the head 100 and flexible/bent handle 200 are integrally formed. Optionally, recesses 116 and 118 may be used to engage and/or release flexible/bent handle 200 from head 100.

Alternatively, head 100 may engage with handle 200 using any other suitable connectors 202, such as via a threaded connection, a mounting element, a joint, and/or any other type of coupling. The coupling between head 100 and handle 200 may fixate the orientation of head 100 with respect to handle 200, such that the movement of head 100 is actuated solely by the user's maneuvering of handle 200. In some embodiments, head 100 may be at least partially rotatable about handle 200.

Optionally, handle 200 may be reusable, or disposable and may be suited for sterilization.

Reference is now made to FIG. 3E which is an illustration of handle 200 of the device of FIG. 3D, according to some embodiments of the invention. Handle 200 may include two portions 200A and 200B and a connector 202. In some embodiments, handle 200 may be a flexible handle made from a flexible material. In some embodiments, the flexibility may be achieved by forming a returnable bending between portions 200A and 200B and/or between portion 200B and connector 202. For example, a spring-like element may connect portions 200A to 200B and/or portion 200B to connector 202.

In some embodiments, handle 200 may be a bent handle having a fixed predetermined angle between portion 200B to connector 202. An enlarged view of connector 202 connected to portion 200B at a fixed angle is illustrated in FIG. 3F. In some embodiments, the angle α is defined as the angle between the central axis x of handle 200 and a connecting surface ‘S’ of connector 202. In some embodiments, the angle α is between 90° to 150°. In some embodiments, angle α is between 100° to 150°, between 110° to 150°, between 115° to 150° and between 120° to 150°.

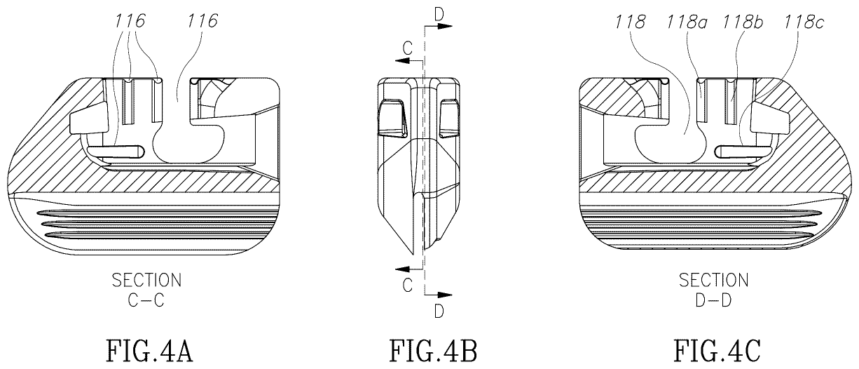

Reference is now made to FIGS. 4A-4C which show three cross-sectional views of head 100 with detailed views of recesses 114, 116, and 118, 118a, 118b and 118c that are configured to engage and/or release flexible/bent handle 200 from head 100. Recess 118 may include one or more rachet-type features that may alternately lock and release flexible/bent handle 200 with head 100.

The following is a method for modifying a tissue, according to an embodiment.

Optionally, the method may be for modifying the corneal epithelium. Optionally, the method may be performed prior to refractive eye surgery, for treating disorders such as myopia, hyperopia, astigmatism, keratoconus or others. The refractive surgery may include, for example, procedures for reshaping the curvature of the cornea, for example using surface ablation methods such as Photorefractive Keratectomy (PRK), Photo Therapeutic Keratectomy (PTK), Laser-Assisted Sub Epithelium Keratomileusis (LASEK), EPI-LASEK, Advanced Surface Ablation (ASA) techniques.

Optionally, the method may be for modifying vaginal tissue, nasal tissue, oral tissue, and the like.

Optionally, the method may comprise positioning a plurality of blades such as two, three, or a higher number of blades on a surface of the tissue. Optionally, the blades are configured on a head of a device as described above. Optionally, the head is coupled to a flexible handle which is manually maneuvered by a user. Optionally, the method may include applying external pressure on a flexible handle connected to an elongated head, for example, flexible/bent handle 200 connected to any one of elongated heads 100 and 401.

Optionally, the method comprises modifying the tissue. Optionally, modifying refers to altering the tissue by peeling/removing the tissue. Optionally, modifying includes reducing the thickness of the tissue. For example, the thickness of the removed layer varies between different portions of the cornea surface, for example, a thicker layer is removed from the center of the cornea, and a thinner layer is removed from the periphery. Alternatively, the thickness of the removed layer is constant and substantially even for the various treated portions of the cornea surfaces. In some embodiments, modifying includes reshaping the epithelium. Optionally, modifying does not affect the Bowman's layer under the epithelium. For example, modifying includes removing at least a portion of the Bowman's layer. In some embodiments, modifying does not cause damage to the stroma.

Optionally, modification is of other superficial tissues, for example, the facial skin, any other skin tissues, the vaginal tissue, oral tissue and the like.

Optionally, modification is obtained by collecting a tissue. In some embodiments, modification is obtained by peeling the tissue. Optionally, peeling is carried out by moving the blades across a surface of the tissue. Optionally, moving comprises stroking type movement, saccadic movement, one-directional movement, two directional movement. In some embodiments, the blades are caused to slide across the tissue.

In some embodiments, the blades are configured to form a slope in the tissue during their movement, for example a slope between a center of the cornea and a circular periphery of the cornea. Optionally, the slope angle ranges between 10-30 degrees, such as 12 degrees, 18 degrees, 25 degrees, or intermediate, larger or smaller angles. A potential advantage of producing a slope by movement of the blades across the cornea surface may include inducing cell growth, which may provide a faster healing rate of the tissue, for example following a refraction procedure. An effect of the slope may include faster regrowth of cells at the bottom of the slope, for example at the cornea center, which may accelerate healing.

Optionally, removed tissue and/or fluid are collected, for example drained into a funnel between the blades.

Optionally, the surface of the tissue is dried. In some embodiments, drying is performed by collecting fluid during movement of the device across the tissue, for example by using one or more absorbing elements, for example as further described herein. Optionally, drying is obtained by draining fluid utilizing capillary action of a funnel defined in between the blades.

Optionally, after removal and/or other modification of the tissue, and/or once the tissue is dry (for example relative to a natural state of the cornea), a refractive surgery is performed.

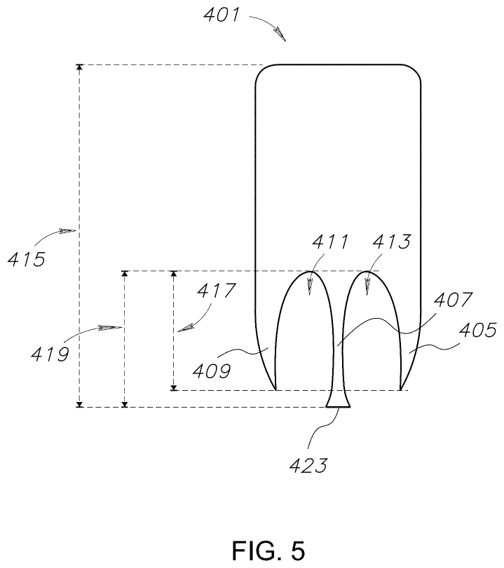

FIG. 5 is a front view of an exemplary head of a device for modifying the tissue, comprising or consisting of three blades, according to some embodiments of the invention.

In some embodiments, head 401 comprises a plurality of blades gaggers, such as gaggers 405 and 409 and at least one blade 407 shown herein, disposed at a distal end of head 401. In some embodiments, at least one blade 407 comprises a distal contacting surface or edge such as contacting surface 423. In some embodiments, two funnels are defined between the walls of the base portions of adjacent the blades and gaggers, a first funnel 411 defined between the base portions of gagger 409 and blade 407, and a second funnel 413 defined between the base portion of gagger 405 and baled 407.

In some embodiments, funnels 411 and 413 extend in a proximal direction, for example extending over ⅛ to ½ of a height 415 of head 401, the height measured between the proximal and distal ends of the head.

In some embodiments, gaggers 405 and 409 and at least one blade 407 are formed with different heights with respect to each other (the height being measured, for example, from a proximal end of the funnels formed between the blades, to a contacting surface of each blade/gagger which engages the tissue). Optionally, a height difference between the blades/gaggers determines a depth of penetration of the blades with respect to a surface of the tissue. In some embodiments, the blades and gaggers are configured so that the gagger limits a depth of penetration of at least one blade with respect to a surface of the tissue. For example, as shown in this figure, the two outermost gaggers 405 and 409 each act as positioning (also “control”) gaggers for middle blade 407, which is double-edged. In some embodiments, a gagger is formed with a height 417 that is shorter than height 419 of middle blade 407, for example, 10%, 20%, 30%, 40%, or intermediate, larger or smaller percentages shorter. Optionally, height 419 of middle blade 407 ranges between, for example, 2-3 mm, such as 2 mm, 2.2 mm. 2.7 mm, or intermediate, longer or shorter heights, and height 417 of gagger 405 is shorter than height 419, by, for example, 0.15-0.2 mm. In some embodiments, the height differences between the blades and gaggers maintain a fixed positioning of the blades with respect to a curved surface of the tissue. A potential advantage of one or more blades configured for limiting a depth of penetration of another blade may include preventing a blade from damaging deeper layers of tissue, for example, layers underneath the epithelium. The fixed positioning may provide additional safety, for example by reducing damage to deeper tissue layers which in turn may be caused by a user applying excessive pressure when maneuvering the device over the tissue. A potential advantage of the blade arrangement may include reducing the effect of a force applied by a user on the depth of the blades within the fluidic layer of the epithelium. Gagger 405 and/or gagger 409, act to stabilize blade 407 on the tissue's surface. Optionally, gagger 405 acts to stabilize blade 407 when peeling is performed in a first direction, and gagger 407 acts to stabilize blade 407 when peeling is performed in an opposite direction. Potentially, even uncontrolled movement of the device across the tissue will not cause substantial damage to deeper tissue layers and may reduce the risk of tearing cornea tissue during peeling.

In some embodiments, a guiding gagger such as gagger 405 defines an entrance angle α of the middle blade 407 with respect to the tissue. Entrance angle α is defined, for example, with respect to a horizontal axis, passing through a point or line formed by one of the edges of contacting surface 423 of blade 407 (the edges are the right and left corners of contacting surface 423 shown in the figure). Optionally, the opening of angle α is set by the positioning of the contact surface of gagger 505 with respect to the horizontal axis. Optionally, angle α ranges between, for example, 38-55 degrees, such as 40 degrees, 47 degrees, 52 degrees or intermediate, larger or smaller angles.

In some embodiments, guiding gaggers 405 and 409 are identical, forming a symmetrical head. Alternatively, gagger 405 is different from gagger 409 in height and/or in a size of the contacting surface or edge and/or in a distance of the contacting surface of the gagger from the contacting surface of middle blade 407. Optionally, a non-symmetrical configuration of the head provides for treating in one direction in a different manner than the other direction, for example peeling a thicker layer of tissue when moving the head in a first direction, and a thinner layer when moving the head in a second direction. Optionally, the arrangement of the blades/gaggers and their respective heights, contacting surfaces, distances between the blades and/or other parameters are selected according to a topography of the tissue's surface.

In some embodiments, the device is moved across the cornea or any other tissue surface, such as the facial skin, the cervix, the throat, and the like to peel the epithelial tissue. Optionally, movement is performed on a plane tangential to curvature of the cornea, for example in one or two directions (left and/or right directions) that are substantially transverse to height 415 of head 401. For the exemplary head shown in this figure, movement to the right will cause the right edge of contacting surface 423 of blade 407 to peel epithelial tissue which will collect within funnel 413, while movement to the left will cause the left edge to peel epithelial tissue which will collect within funnel 411. A movement to the right is defined by the positioning of gagger 405 with respect to gagger 407 and with respect to the cornea or any other tissue surface, while movement to the left is defined by the positioning of gagger 409 with respect to blade 407 and with respect to the cornea or any other tissue surface. Optionally, the peeled tissue is collected on the side walls of blade 407 as the device is advanced across the corneal surface, optionally accumulating within the draining funnels 411 and 413.

In some embodiments, the epithelial layer may be removed from surfaces, such as, the facial skin, the cervix, the throat, and the like. Therefore, epithelial tissue peeled/removed from these surfaces may be accumulated within the draining funnels 411 and 413.

In some embodiments, the walls of the blades which define funnels 411 and 413 comprise one or more slots (not shown in this figure). The slits act to increase the total surface area of the walls that define the funnel and may increase the capillary forces of the funnel, causing fluid and/or removed tissue to adhere to the funnel and to be sucked in the proximal direction and away from the cornea surface. In some embodiments, a funnel is conical, comprising an opening which widens in a proximal direction. A potential advantage of the conical configuration may include causing fluid and/or tissue to adhere to the funnel walls and be drawn up into the funnel at the contact area between the cornea and the funnel, in which the opening is of a relatively small diameter. The capillary action may decrease as the funnel widens in a proximal direction.

In some embodiments, gaggers 405, 409, and/or blade 407 may comprise a biocompatible polymer. Alternatively, in some embodiments, the blades are composed of metal. Optionally, the metal blades are manufactured with a high surface finish, to reduce inflammation and/or an engraving effect on the cornea. In some embodiments, the material of which the blades are composed of or are coated with is hydrophilic, for increasing the effect of collecting fluid. In some embodiments, gaggers 405, 409, and/or blade 407 may comprise a polymer having a shore hardness of between 20 to 90 SHD.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

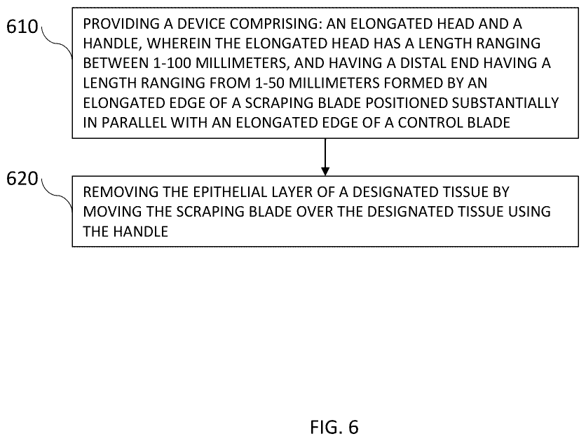

Reference is now made to FIG. 6 which is a flowchart of a method for selectively peeling a layer of a superficial tissue. In step 610, a device configured to selectively peel the layer (e.g., the epithelial layer) may be provided. In some embodiment, the device may include elongated head 100/401 and a handle 200.

In step 620, the layer of a designated superficial tissue may be removed by moving the blade over the designated tissue using the handle.

In some embodiments, the superficial tissue is a portion of a facial skin. In such case, removing the layer may further include at least one of: removing age spot, removing shallow lesion, removing post-surgery scars and the like.

In some embodiment, the superficial tissue is the cervix. In some embodiments, the superficial tissue is the esophagus (throat). In some embodiments, removing the layer further includes at least one of, collecting the removed layer for pathology sampling and collecting the removed layer for smear squamous cell sampling, such as cervical smear, throat swab and the like. In some embodiments, collecting epithelial layer is by accumulating the layer in a cavity between guiding gagger 102/405 and the blade 104/407, as discussed herein above with respect to FIG. 6. Therefore, tissue peeled/removed from these surfaces may be accumulated within cavities such as draining funnels 411 and 413. In such case device 100/401 further includes a second guiding gagger 409, and blade 407 is a double-sided blade, and the two guiding gaggers 405 and 409 are disposed on either side of double-sided blade 407.

In some embodiments, at least one of the elongated edges is covered with a functional coating, selected from: a lubrication material, a medication, nutritive material, moisturizing material, and an antiseptic material. In some embodiments, the device is a disposable device and wherein the scraping blade is made from a polymeric material having a shore hardness of between 20 to 90 SHD, as discussed herein above.

Some additional embodiments of the invention may be directed to a ‘kit’ which may include a device according to any one of the embodiments disclosed herein above (e.g., a device comprising elongated heads 100/401 and handle 200) and least one formulation. In some embodiments, the formulation may include active and nonactive materials to be provided to the pealed surface. For example, if the peel surface is the facial skin the formulation may include at least one of, a moisturizing component, a vitamin selected from, E, B1, hyaluronic acid, retinol, aromatic oils, plant extracts, and the like. In yet another example, if the peeled surface is the cervix, the formulation may include at least one of, a moisturizing component, an antibacterial component, antigentile fungus component and the like.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A device comprising:
an elongated head and a flexible handle,
wherein the flexible handle comprises at least one component configured to bend the flexible handle upon application of external pressure and return the flexible handle to original position upon releasing of the external pressure.

2. The device of claim 1, wherein the at least one component is selected from: at least a portion of the flexible handle made from an elastic material, a spring, and a joint.

3. (canceled)

4. The device of claim 1, wherein the flexible handle comprising a connector for detachably connecting the flexible handle to the elongated head.

5. The device of claim 1, wherein the head is disposed with one or more recesses that are configured to engage with the flexible handle, and wherein the recesses are disposed at a proximal end of the head and engaging with the flexible handle comprises having no contact with the distal edges of blades included in the elongated head.

6. The device of claim 1, wherein the elongated head comprises one or more blades positioned substantially in parallel with at least one guiding gagger and wherein the elongated head has a length ranging between 1-100 millimeters, and having a distal end having a length ranging from 1-50 millimeters formed by an elongated edge of the one or more blades.

7. The device of claim 6, wherein the elongated edge of at least one blade is covered with a functional coating, selected from: a lubrication material, a medication, nutritive material, moisturizing material, and an antiseptic material.

8. The device of claim 6, further comprising another guiding gagger, wherein the blade is a double-sided blade, and wherein the guiding gaggers are disposed on either side of the one or more blades.

9. A method for modifying a tissue, comprising:
applying external pressure on a flexible handle connected to an elongated head, wherein the flexible handle comprises at least one component configured to bend the flexible handle upon application of external pressure and return the flexible handle to its original position upon releasing of the external pressure.

10. The method of claim 9, wherein applying the external pressure is conducted in motion while moving the elongated head over the tissue.

11. The method of claim 9, wherein the at least one component is selected from at least a portion of the flexible handle made from an elastic material, a spring, and a joint.

12. The method of claim 9, wherein the peeled portion of the tissue is the corneal epithelium that does not include the Bowman's layer.

13. The method of claim 9, wherein the peeled portion of the tissue is a portion of one of, vaginal tissue, an oral tissue, and a nasal tissue.

14. The method of claim 9, further comprising collecting any of peeled tissue and released residue in a funnel of the head.

15. The method of claim 14, wherein the released residue is a liquid.

16. The method of claim 14, wherein collecting comprises absorbing with an absorbent material at least partially coating the funnel.

17. The method of claim 9, further comprising orienting the elongated head with respect to the tissue to define the penetration depth of an elongated edge of one or more blades of the elongated head, into the tissue.

18. (canceled)

19. (canceled)

20. (canceled)

21. The method of claim 17, wherein applying the external pressure comprises pressing the elongated edge of the one or more guiding gaggers onto the tissue to flatten the surface of the tissue, and enclosing a portion of the flattened tissue within the gap, thereby peeling the tissue at a uniform thickness.

22. The method of claim 9, further performing any of: a refractive eye surgery, treating a myopia disorder, treating a hyperopia disorder, treating a astigmatism disorder, and treating a keratoconus disorder subsequent to the peeling step.

23. A device comprising:
an elongated head and a bent handle bent at a predetermined angle between a central axis of a portion of the handle configured to held by a user and a connecting surface of a connector connecting the elongated head,
wherein the predetermined angle is 90-150 degrees with respect to the head and the blades.

24. (canceled)

25. The device of claim 23, wherein the connector is configured to detachably connect the flexible handle to the elongated head.

26.-37. (canceled)
